(19) United States
(12) Patent Application Publication
(10) Pub. No.: US 2023/0073716 A1
(43) Pub. Date: Mar. 9, 2023

(54) STORAGE CONTAINER WTIH FILTRATION SYSTEM

(71) Applicant: PHILIP MORRIS PRODUCTS S.A., Neuchâtel (CH)

(72) Inventors: Rui Nuno Batista, Morges (CH); Ricardo Cali, Mannheim (DE); Thomas Edmund McGrath, St-Légier-La Chiésaz (CH); Riccardo Riva Reggiori, St-Sulpice (CH); Maria Miguel Matias Serrador, Lausanne (CH); Cheng Peng, Hong Kong (HK); Hanna Ulrika Furustrand Tafin Djoko, Boudry (CH); Tobias Lipp, Santiago de Compostela (ES)

(73) Assignee: PHILIP MORRIS PRODUCTS S.A., Neuchâtel (CH)

(21) Appl. No.: 17/795,189

(22) PCT Filed: Jan. 29, 2021
(86) PCT No.: PCT/IB2021/050744

§ 371 Date: Jul. 25, 2022

(30) Foreign Application Priority Data
Jan. 30, 2020 (EP) 20154726.2

(51) Int. Cl. D06F 58/10; D06F 58/22; D06F 34/26; D06F 34/05; B01D 46/56; B01D 46/46; B01D 47/00; B01D 46/00; B01D 46/42; B01D 50/60; D06F 19/00; D06F 35/00; A61L 2/26; A61L 2/10; A61L 2/20; A47B 61/00

(52) U.S. Cl. CPC D06F 58/10; D06F 58/22; D06F 34/26; D06F 34/05; B01D 46/56; B01D 46/46; B01D 47/00; B01D 46/0032; B01D 46/429; B01D 50/60; D06F 19/00; D06F 35/001; A61L 2/26; A61L 2/10; A61L 2/202; A47B 61/003; D06F 2103/28

(57) ABSTRACT

A storage container (100) comprising a housing (110) containing a storage chamber (112) isolated from an outside environment (10). The housing (110) defines a housing inlet (130) and a housing outlet (140). The storage container (100) is configured to clean air in the outside environment (10). The storage container (100) comprises a first filtration assembly (360) coupled to the housing (110) about the housing outlet (140). The first filtration assembly (360) has a first filtration inlet (364) configured to receive air from the storage chamber, a first filtration outlet (366) configured to release air into the outside environment (10), and first filter media (368) between the first filtration inlet (364) and the first filtration outlet (366).

The present disclosure relates to a storage container having a filtration system to purify air. More particularly, the present disclosure relates to a storage container such as a wardrobe having a filtration system that cleans the air in environment outside the storage container. The filtration system may also clean items such as clothing stored within the storage container.

A number of types of storage containers such as wardrobes that have functionality to clean stored clothes exist or have been proposed in the art. Some of these storage containers generally have a hanging area within which clothes may be hung for storage. These storage containers may have a wide variety of components that facilitate cleaning clothes directly or indirectly (for example, cleaning the environment of the storage container's interior, which surrounds the clothes). Various types of storage containers incorporate air filtration to purify the air inside of the storage container. Some storage containers incorporate components such as ultraviolet light sources, steamers, and dehumidifying components (such as an air blower or a dehumidifier), as well.

One Chinese published patent application, CN108741717, discloses a smart wardrobe having an inlet pipe equipped with a filter box to block dust from entering the storage container. Another Chinese published patent application, CN108835922A, discloses an air purifier to purify the air already inside of a wardrobe. The wardrobe also incorporates an ultraviolet sterilization lamp and a dehumidifier. A dehumidifier prevents moisture from permeating into clothes to cause mildew and the ultraviolet sterilization lamp sterilizes the interior of the wardrobe.

According to aspects of the present invention, there is provided a storage container comprising a housing containing a storage chamber isolated from an outside environment. The housing defines a housing inlet and a housing outlet. The storage container has a filtration system configured to clean the air in the outside environment. The filtration system has a first filtration assembly coupled to the housing about the housing outlet, wherein the first filtration assembly has a first filtration inlet configured to receive air from the storage chamber, a first filtration outlet configured to release air into the outside environment, and first filter media between the first filtration inlet and the first filtration outlet. The storage container is configured to clean the air in the outside environment.

According to aspects of the present invention, a storage container may comprise a housing containing a storage chamber. The storage chamber may be isolated from an outside environment. The housing may define a housing inlet. The housing may define a housing outlet. The storage container may have a first filtration assembly. The first filtration assembly may be coupled to the housing about the housing outlet. The first filtration assembly may have a first filtration inlet. The first filtration inlet may be configured to receive air from the storage chamber. The first filtration assembly may have a first filtration outlet. The first filtration outlet may be configured to release air into the outside environment. The first filtration assembly may have first filter media between the first filtration inlet and the first filtration outlet. The storage container may be configured to clean the air in the outside environment.

Configurations such as described above may advantageously allow the storage container to simultaneously function as an air purifier for the room within which the storage container is placed and a cleaner of items within the storage container. For example, the first filtration assembly of the storage container may purify (clean) the air in the storage container and may release that air to the environment outside of the storage container. This functionality may eliminate the need to have a separate air purification device in the room, which may save space, money, and resources.

The storage container may comprise a second filtration assembly coupled to the housing about the housing inlet. The second filtration assembly may comprise a second filtration inlet. The second filtration inlet may be configured to receive air from the outside environment. The second filtration assembly may comprise a second filtration outlet. The second filtration outlet may be configured to release air into the storage chamber. The second filtration assembly may comprise second filter media. The second filter media may be between the second filtration inlet and the second filtration outlet. The second filtration assembly may advantageously filter air that is brought into the storage container from outside the storage container, such as in an outside room, so that the outside air is cleaned and, simultaneously, items within the storage container are exposed to clean air relative to the outside room.

The first filtration assembly may comprise an air washer. The air washer may advantageously help purify the air passing from inside the storage container to outside the storage container. The air washer may advantageously simultaneously clean the air from the outside environment and clean items inside the storage container.

The storage container may comprise a dehumidifier. The dehumidifier may have a dehumidifier inlet. The dehumidifier inlet may be configured to receive air from the storage chamber. The dehumidifier may reduce moisture in the air of the storage chamber, which may advantageously slow or cease microbial growth. The dehumidifier may simultaneously and advantageously reduce moisture in the outside environment and in items stored in the storage chamber. The dehumidifier may have a liquid conduit fluidly coupling the dehumidifier and the first filtration assembly. Such a configuration may advantageously conserve water. Such a configuration may save a user time that would otherwise be spent refilling the reservoir of the air washer. Such a configuration may also reduce complexity of the system by eliminating the need for a water conduit to flow water to the air washer through the storage container.

The storage container may comprise a clothes hanging structure configured to receive hanging clothing. The clothes hanging structure may advantageously allow for clothing storage that prevents wrinkles in the clothing through the force of gravity on the clothing. The clothes hanging structure may also advantageously allow for gravity-assisted cleaning of the clothing, where particles may be removed from the clothing with gravity. The hanging structure may comprise a hanging rod extending across at least a portion of the storage chamber. The hanging rod may be configured to receive hangers of clothing.

The storage container may comprise a clothes cleaning system in the storage chamber. The clothes cleaning system may be configured to execute cleaning functions within the storage chamber. A clothes cleaning system may advantageously allow for clothes in the storage container to be cleaned while being stored. Components of the clothes cleaning system may advantageously simultaneously clean the air from the outside environment. The clothes cleaning system may comprise a vibration generator mechanically coupled to a clothes hanging structure. The vibration generator may advantageously shake debris such as dust, dirt, and other particles from the clothing and allow gravity to transport the debris away from the clothing. The clothes cleaning system may comprise a debris collector positioned below the clothes hanging structure. The debris collector may advantageously collect the debris. The clothes cleaning system may comprise a vertical air current generator. The vertical air current generator may be configured to generate downward airflow through the storage chamber to assist in moving debris to the debris collector. The vertical air current generator may be configured to generate laminar airflow. The vertical air current generator may advantageously transport debris downward to the debris collector.

The clothes cleaning system may comprise an antimicrobial unit disposed in the storage chamber. The antimicrobial unit may advantageously neutralize or limit the growth of microbes within the storage chamber, such as microbes that may be on items, such as clothing, stored in the storage container. The antimicrobial unit may advantageously simultaneously neutralize or limit the growth of microbes within the air from the outside environment. The antimicrobial unit may comprise an ultraviolet (UV) light source disposed in the storage chamber. The antimicrobial unit may comprise an electrostatic precipitator disposed in the storage chamber. The clothes cleaning system may comprise an airflow conduit extending through a length of the storage chamber. The airflow conduit may have a conduit inlet configured to receive air from the second filtration outlet. The airflow conduit may have a plurality of openings that cumulatively define a conduit outlet along the length of the storage chamber. The airflow conduit may advantageously direct purified air to the storage chamber. The purified air may thus contact items, such as clothing, within the storage chamber. The plurality of openings may advantageously direct purified air to contact stored items, such as clothing, at multiple locations to help dislodge debris.

The second filtration assembly of the storage container may comprise an electrostatic precipitator. The electrostatic precipitator may advantageously be configured to clean the air from the outside environment which is used to clean items in the storage chamber. The second filtration assembly may comprise a second filtration assembly monitoring system. The second filtration monitoring system may be configured to receive and monitor air from the second filtration inlet. The storage container may comprise a second filtration assembly communication module. The second filtration assembly communication module assembly may be coupled to the storage container. The second filtration assembly communication module may be in data communication with the second filtration assembly monitoring system. The second filtration assembly communication module may comprise a wireless communication component. The wireless communication component may be configured to communicate with a user device.

The storage container may comprise an ozone sensor disposed in the storage chamber. The ozone sensor may be in operative communication with the first filtration assembly. The ozone sensor may be configured to disable operation of the first filtration assembly upon sensing a predefined level of ozone. The ozone sensor may be configured to disable operation of the first filtration assembly upon sensing a minimum level of ozone. Such a configuration advantageously prevents the ozone in the storage chamber from being released into the outside environment.

The storage container may be configured to clean the air in the environment outside of the storage container and clean items stored within the storage container simultaneously. The storage container may be configured to alternately clean the air in the environment outside of the storage container and clean items stored within the storage container. In various examples, the storage container may be configured to independently clean items stored within the storage container and clean the air in the outside environment. In various examples the storage container may sequentially clean items stored within the storage container and clean the air in the outside environment. In various examples, cleaning items stored within the storage container and cleaning the air in the outside environment may overlap during some time periods. For example, the storage container may start with cleaning the air in the outside environment and then clean the items stored inside the storage container, and then continue to clean the items in the storage container after the outside air is no longer being cleaned (such as when the outside air does not require further purification).

The storage container may comprise a first air monitoring system in gaseous communication with the storage chamber. The first air monitoring system may be configured to identify gaseous constituents in the storage chamber. The first air monitoring system may be in operative communication with the clothes cleaning system. The first air monitoring system may be configured to execute cleaning functions of the clothes cleaning system in accordance with the gaseous constituents identified by the first air monitoring system. Such a configuration may advantageously allow the storage container to adapt cleaning functionality to be appropriate to the conditions inside the storage chamber.

The storage container may comprise a first filtration assembly communication module coupled to the storage container. In examples incorporating a first air monitoring system, the first filtration assembly communication module may be in data communication with the first air monitoring system. The first filtration assembly communication module may comprise a wireless communication component configured to communicate with a user device. Such a configuration may advantageously inform a user of system status, air quality status, or both system status and air quality status. In examples incorporating a second filtration assembly, the second filtration assembly may comprise a second air monitoring system. The storage container may be electrically connectible to a home utility control system for selective operation of the storage container through the home utility control system. Such a configuration may advantageously allow the storage system to be operated at a centralized location from which other home utilities are operated by a user.

According to other aspects of the present invention, a method of simultaneously cleaning air in an outside environment and cleaning items stored in a storage chamber is provided. Chamber air is passed from a storage chamber of a housing through a first filtration assembly. The chamber air is passed through a housing outlet of the housing. Outside air is passed from outside a housing through a housing inlet of the housing. The outside air is passed through a second filtration assembly into the storage chamber of the housing.

According to aspects of the invention, a method for simultaneously cleaning air in an outside environment and cleaning items stored in a storage chamber may comprise passing chamber air from a storage chamber of a housing through a first filtration assembly. The method may comprise passing chamber air through an outlet of the housing. The method may comprise passing outside air from outside the housing through an inlet of the housing. The method may comprise passing the outside air through a second filtration assembly into the storage compartment of the housing.

Methods such as described above may advantageously filter the air entering the storage container to simultaneously create a relatively cleaner environment in the storage chamber and in the outside environment. The method may advantageously allow the storage container to function as an air purifier for the room within which the storage container is placed. This functionality may eliminate the need to have a separate air purification device in the room, which may save space, money, and resources.

The method may comprise dehumidifying air inside of the storage chamber with a dehumidifier. The dehumidifier may collect water from the dehumidifying step. The first filtration assembly may comprise an air washer. The water may be transferred from the dehumidifier to the first filtration assembly. The first filtration assembly may use the transferred water to filter the chamber air. Such a method may advantageously conserve water. A method employing such a configuration may save a user time that would otherwise be spent refilling the reservoir of the air washer. Such a method may also reduce complexity of the system by eliminating the need for a water conduit to flow water to the air washer through the storage container.

The method may comprise vibrating a hanging rod disposed in the housing. Such a method may advantageously allow for removing debris from the clothing. The method may comprise collecting debris in a debris collector positioned below the hanging rod.

The method may comprise exposing the storage chamber to an antimicrobial unit disposed within the chamber. The antimicrobial unit may advantageously neutralize or limit the growth of microbes within the storage container, such as microbes that may be on items, such as clothing, stored in the storage container. The antimicrobial unit may advantageously neutralize or limit the growth of microbes of the air within the storage container. The antimicrobial unit may comprise UV light from a UV light source. The method may comprise directing outside air from the second filtration assembly through an airflow conduit and out of a conduit outlet. The conduit outlet may be defined by a plurality of openings positioned along a length of the storage chamber. The conduit outlet may advantageously direct purified air to the storage chamber, help dislodge debris, or both direct purified air to the storage chamber and help dislodge debris, as discussed above regarding the storage container.

The method may comprise generating ozone. The ozone may be generated by the second filtration assembly. The ozone may advantageously deodorize, disinfect, or deodorize and disinfect the storage chamber. The ozone may advantageously deodorize, disinfect, or deodorize and disinfect items, such as clothes, within the storage chamber, as well as the air within the storage chamber. The method may comprise detecting ozone by an ozone sensor. The ozone sensor may be disposed in the storage chamber. The ozone sensor, or control electronics operably coupled to the ozone sensor, may disable operation of the first filtration assembly upon detection of a minimum level of ozone. This step may have the advantage of reducing or preventing release of ozone into the outside environment. The ozone sensor, or control electronics operably coupled to the ozone sensor, may send data sensed by the ozone sensor to a user device such as a laptop or a smartphone.

The method may comprise identifying gaseous constituents in the storage chamber by a first air monitoring system and executing cleaning functions in accordance with the constituents identified by the first air monitoring system. Such a step may advantageously increase efficiency by preventing unnecessary cleaning functions.

According to other aspects of the present invention, there is provided a method of filtering air. Chamber air in a storage chamber of a housing is dehumidified and water is collected from the dehumidifying. Water is transferred from the dehumidifier to an air washer coupled to a housing outlet of the housing. The chamber air is washed in the storage chamber by the air washer using the transferred water. The air is expelled from the storage chamber through the housing outlet.

According to other aspects of the present invention, there is provided a method of filtering air. The method may comprise dehumidifying chamber air in a storage chamber of a housing and collecting water from the dehumidifying. The method may comprise transferring the water from the dehumidifier to an air washer coupled to a housing outlet of the housing. The method may comprise washing the chamber air in the storage chamber by the air washer using the transferred water. The method may comprise expelling air from the storage chamber through the housing outlet.

Such a method may advantageously conserve water by using the water collected by the dehumidifier to filter the air. Such a configuration may save a user time that would otherwise be spent refilling the reservoir of the air washer. Such a configuration may also reduce complexity of the system by eliminating the need for a water conduit to flow water to the air washer through the storage container. The method may be used to advantageously clean the outside air as well as items stored in the storage container.

The method may further comprise passing the chamber air through a first filtration assembly comprising the air washer. The method may further comprise passing outside air from outside the housing through a housing inlet of the housing and a second filtration assembly into the storage chamber of the housing. Such a step advantageously provides a relatively clean environment inside the storage chamber. Such a step advantageously cleans the outside air.

The method may further comprise vibrating a hanging rod disposed in the housing. Such a method may advantageously allow for removing debris from the clothing. The method may further comprise collecting dust debris in a dust debris collector positioned below the hanging rod.

The method may further comprise exposing the storage chamber to an antimicrobial unit disposed within the chamber. The antimicrobial unit may comprise UV light from a UV light source.

The method may further comprise directing outside air from the second filtration assembly through an airflow conduit and out of a conduit outlet. The conduit outlet may be defined by a plurality of openings positioned along a length of the storage chamber. The conduit outlet may advantageously direct purified air to the storage chamber, may help dislodge debris, or may direct purified air to the storage chamber and help dislodge debris, as discussed above regarding the storage chamber.

The method may further comprise generating ozone. The ozone may be generated by the second filtration assembly. The ozone may advantageously deodorize, disinfect, or deodorize and disinfect the storage chamber. The ozone may advantageously deodorize, disinfect, or deodorize and disinfect items, such as clothes, within the storage chamber, as well as the air in the storage chamber. The method may further comprise detecting ozone by an ozone sensor. The ozone sensor may be disposed in the storage chamber. The ozone sensor, or control electronics operably coupled to the ozone sensor, may disable operation of the first filtration assembly upon detection of a minimum level of ozone. This step may have the advantage of preventing the release of ozone into the outside environment.

The method may comprise identifying gaseous constituents in the storage chamber by a first air monitoring system and executing cleaning functions in accordance with the constituents identified by the first air monitoring system. Such a step may advantageously increase efficiency by preventing unnecessary cleaning functions.

The terms used herein will have their generally accepted definitions unless otherwise defined herein.

As used herein, the singular forms “a,” “an,” and “the” also encompass embodiments having plural referents, unless the content clearly dictates otherwise.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the disclosure, including the claims.

Any direction referred to herein, such as “top,” “bottom,” “left,” “right,” “upper,” “lower,” and other directions or orientations are described herein for clarity and brevity but are not intended to be limiting of an actual device or system. Devices and systems described herein may be used in a number of directions and orientations.

The phrase “air washer” is a device that purifies air by exposing the air to water. For example, an air washer may spray the air with water. In another example, an air washer may bubble air through water. Other configurations are also possible.

A “dehumidifier” is a component that reduces humidity in air.

An “electrostatic precipitator” is a device that filters particles from air by applying an electrostatic charge to an air stream, which results in charging the particles suspended in the air stream, and then collecting the charged particles to remove them from the air, such as with a collection electrode.

The phrase “filter media” or “filtration media” is used herein to mean a physical substance that is used to filter. Filter media or filtration media may incorporate filter textiles, particulates, membranes, screens, liquids, and combinations thereof.

A “microbe” is a microorganism or a microbial agent that may invade and replicate with a cell of an organism. A microbe may include, but is not limited to, bacteria, viruses, protozoa, archaea and fungi.

A “home utility control system” is a system that is configured for selective electrical communication with home utilities such as a light, thermostat, blinds, a refrigerator, and an audio system, as examples. A home utility control system may be able to turn home utilities on and off. A home utility control system may be able to regulate home utilities such as temperature, volume, and brightness.

Various storage containers consistent with the present disclosure are configured to purify a storage chamber of the storage container to simultaneously clean items stored therein and clean the air in the outside environment. The outside environment may be a room within which the storage container is situated.

The present disclosure also relates to storage containers that may use resources such as water more efficiently than previous storage containers. For example, storage containers may collect water for dehumidification purposes, and then use the collected water for air filtration. Such a configuration may conserve water.

Storage containers consistent with this disclosure are generally configured to store items. Storage containers may be configured to store items such as articles of clothing, shoes, accessories, and the like. The storage container may have a housing configured to isolate the storage chamber from the outside environment, meaning that the container housing is configured to prevent unrestricted airflow between the storage chamber and the outside environment. The housing may substantially surround the storage chamber. Various aspects of the housing and storage chamber are described in more detail below.

Some storage containers that are configured to clean the air of the outside environment may be configured to intake air from the outside environment, clean the air, and then release the cleaned air back into the outside environment. The air may be cleaned with a first filtration assembly that filters the air. In such examples, a housing of the storage container may define a housing inlet, a housing outlet, and the storage chamber, through which air from the outside environment is circulated. Air from the outside environment may pass into the storage chamber through the housing inlet. Air from the storage chamber may pass into the outside environment through the housing outlet. Air circulating through the housing inlet, housing outlet, and the storage chamber passes through the first filtration assembly before being released into the outside environment.

The storage container may have any suitable capacity to clean the air from particles like dust, smoke, and pollen. In some embodiments, the storage container has a capacity to deliver cleaned air at a rate of 1300 cubic meters per hour or greater. In some embodiments, the storage container has a capacity to deliver cleaned air at a rate of 1350 cubic meters per hour or greater. The capacity to deliver clean air may be measured using a standard for measuring clean air delivery rate (CADR). An example of such a standard is ANSI/AHAM AC-1-2019.

The first filtration assembly is configured to filter air and may release the filtered air into the outside environment. The first filtration assembly may be configured to filter the air from the storage chamber. The first filtration assembly may have a first filtration inlet that is configured to receive air from the storage chamber. The first filtration assembly may have a first filtration outlet that is configured to release air into the outside environment. The first filtration assembly may have first filter media between the first filtration inlet and the first filtration outlet. As such, air from the storage chamber may pass through the first filter media before passing through the first filtration outlet. In this way, cleaned air is introduced into the outside environment.

Preferably, the first filtration assembly is coupled to the housing about the housing outlet. In some embodiments the first filtration assembly is coupled to the housing within the storage chamber, such that the first filtration outlet is upstream of the housing outlet. In some other embodiments the first filtration assembly is coupled to the housing outside of the storage chamber, such that the first filtration outlet is downstream of the housing outlet. In some embodiments the first filtration outlet and the housing outlet overlap.

Advantageously, the first filtration assembly of the storage container may purify the air in the storage container and release that air to the outside environment. This functionality may eliminate the need to have a separate air purification device in the room, which may save space, money, and resources.

The first filtration assembly may have a variety of different configurations and components. In some embodiments, the first filtration assembly incorporates an air washer. In such embodiments, the first filter media may be liquid such as water or a water solution. In some embodiments, the first filtration assembly has first filter media that is a textile. Example textiles include, but are not limited to, woven or knit fibrous material where the fibers can be cellulosic fibers, polymeric fibers, or glass fibers, and combinations of cellulosic fibers, polymeric fibers and glass fibers. In some embodiments, the first filtration assembly has first filter media that is a particulate filter media. The first filtration assembly may also incorporate components that are configured to purify the air such as, for example, a UV light source.

In some examples, the storage container may additionally have a second filtration assembly coupled to the housing. The second filtration assembly may be coupled to the housing about the housing inlet. The second filtration assembly may have a second filtration inlet, a second filtration outlet and second filter media between the second filtration inlet and the second filtration outlet. The second filtration inlet may be configured to receive air from the outside environment. The second filtration outlet may be configured to release air into the storage chamber. In such configurations, air from the outside environment passes through the second filter media before passing through the second filtration outlet.

The second filtration assembly may have a variety of different configurations and components. In some embodiments, the second filtration assembly incorporates one or more filter medias. The second filter media may include, for example, a carbon filter media or HEPA (high efficiency particle air) filter media. In some examples the second filter media may include water, such as where the second filtration assembly incorporates an air washer. The second filtration assembly may also incorporate other components that are configured to purify the air such as, for example, a UV light source or an electrostatic precipitator. The air purification of the second filtration assembly advantageously exposes the storage chamber and the items within the storage chamber to relatively clean air compared to the air from the outside environment.

The housing of the storage container may have a variety of different configurations consistent with current technology. In some embodiments, the housing has a floor, a ceiling, and at least one sidewall extending between the base and the ceiling. In some embodiments the housing may omit a floor and a floor of the environment within which the storage container is situated may form the floor of the storage container. The at least one sidewall forms a length of the storage chamber between the base and the ceiling. The at least one sidewall surrounds the storage chamber. In some examples, the at least one sidewall may form a tubular structure such that the sidewall has a circular cross-section about the storage chamber, where the cross-section is perpendicular to the length of the sidewall. In some other examples, the at least one sidewall may be multiple sidewalls that intersect along joints.

The housing may have a user access point from which to access the contents of the storage chamber. The user access point may be a container door coupled to the housing that is configured to selectively obstruct an access point opening defined by the housing. The access point opening may be an opening defined in a sidewall of the storage chamber. In some examples, the access point opening may be an opening defined in the ceiling of the housing. The container door may be slidable, pivotable, or extendable across the opening defined storage chamber to selectively isolate the storage chamber from the outside environment. When in a closed position, the container door may restrict air circulation between the storage chamber and the outside environment. In some embodiments the storage container may have multiple user access points.

The storage container has at least one storage chamber. In some examples, the storage chamber has multiple storage sections. In such examples, one or more divider walls within the storage chamber separate the storage sections. The divider walls may extend between sidewalls, for example. In some embodiments a first divider wall may extend between a sidewall and a second divider wall. The storage sections may be isolated from each other to limit airflow between the sections. In some embodiments, the storage sections may be in airflow communication. In some embodiments, one or more storage sections may define shelving to receive items. One or more storage sections may be used for storing hanging clothes. In some embodiments, one or more storage sections may have one or more drawers or cabinets configured to enclose stored items within the storage chamber. Any combination of storage sections is contemplated. In some embodiments the storage chamber is limited to a single storage section.

The storage container may incorporate a clothes hanging structure to receive hanging clothing. The clothes hanging structure may be one or more hooks configured to receive hanging clothing. The hooks may, for example, be coupled to the storage container within the storage chamber. The hooks may be coupled to the housing or to a divider wall within the storage chamber. In examples, the clothes hanging structure may be a hanging rod extending across at least a portion of the storage chamber. The hanging rod may be configured to receive hangers of clothing. The hanging rod may extend across the storage chamber. The hanging rod may be perpendicular to the length of the storage chamber. The hanging rod may extend across a storage section of the housing or may extend across multiple storage sections. In embodiments incorporating a hanging rod, the hanging rod may extend from a first sidewall of the housing to a second sidewall of the housing. The first sidewall may be opposite the second sidewall or may intersect the second sidewall. In some embodiments the hanging rod extends between divider walls within the housing. In some embodiments the hanging rod extends from a sidewall of the housing to a divider wall of the housing.

The storage container may be configured to clean items stored in the storage chamber. This may be accomplished through a variety of approaches. The storage container may have a clothes cleaning system within the storage chamber that is configured to execute cleaning functions in the storage chamber. The cleaning functions may clean the items in the storage chamber. Some cleaning functionality may simultaneously clean the air in the storage chamber. The clothes cleaning system may have a variety of different components and combinations of components. Example components of clothes cleaning systems will now be described.

In some embodiments incorporating a clothes hanging structure, the clothes cleaning system may include a vibration generator. The vibration generator may be mechanically coupled to the clothes hanging structure. In some embodiments the vibration generator is configured to generate vibrations and transmit the vibrations to the clothes hanging structure. In some embodiments the vibration generator is configured to generate ultrasonic vibrations. The vibrations are transmitted to the clothes hanging structure, which results in vibration of the clothes hanging on the clothes hanging structure. The vibration of the clothes may result in advantageously shaking debris from the clothing.

In some embodiments incorporating a vibration generator, the storage container has a debris collector. The debris collector may be positioned to receive shaken debris from the clothing on the clothes hanging structure. In various embodiments, the debris collector is positioned vertically below the clothes hanging structure such that gravity transports the shaken debris from the clothing to the debris collector. In some embodiments, the debris collector is positioned directly below the clothes hanging structure such that there are no intervening components between the clothes hanging structure and the debris collector.

The clothes cleaning system may have an airflow conduit that is configured to transmit purified air to a portion of the storage chamber, such as a section of the storage chamber. The airflow conduit may extend through a portion of the storage chamber. The airflow conduit may have a conduit inlet configured to receive filtered air. In some embodiments, the airflow conduit is configured to receive filtered air from the second filtration outlet. The airflow conduit may have a conduit outlet. The conduit outlet may be cumulatively defined by a plurality of openings along a portion of the length of the airflow conduit. The plurality of openings may be defined along a length of the storage chamber. In examples, the plurality of openings defining the conduit outlet extend along a substantial portion of the length of a section of the storage chamber. A portion of the airflow conduit may extend substantially parallel to the length of the storage chamber. In some embodiments, the airflow conduit may extend through a section of the storage chamber that has a clothes hanging structure. In embodiments incorporating a hanging rod, a portion of the airflow conduit may extend perpendicularly to the hanging rod.

In various embodiments, the conduit outlet is selectively openable. The conduit outlet may advantageously allow for selective ventilation of a portion of the storage chamber, such as a section of the storage chamber. In some embodiments the conduit outlet may be opened and closed by a user. In some embodiments, the conduit outlet may be operably coupled to suitable control electronics, such as an antenna, to permit receive instructions from a remote device, such as a smartphone, to open or close. In some embodiments the conduit outlet is selectively opened and closed autonomously by a controller of the storage container, various embodiments of which are described below. In some embodiments pulsed air may be released through a plurality of openings defining the conduit outlet. Pulsed air may advantageously shake debris from items adjacent the conduit outlet.

The airflow conduit may extend between the second filtration assembly and the housing outlet. In some embodiments where an airflow conduit extends through a section of the storage chamber, the airflow conduit may extend from a dividing wall to a position adjacent to the housing outlet or, more particularly, adjacent to the first filtration assembly. The airflow conduit may define a secondary outlet adjacent to the first filtration assembly. As such, air enters the airflow conduit through the conduit inlet and exits the airflow conduit through the secondary outlet. Air may also exit the airflow conduit through the plurality of openings, when the plurality of openings are at least partially unobstructed. The air exiting the airflow conduit may pass through the first filtration assembly to the outside environment of the storage container. In some embodiments the secondary outlet may be omitted.

In various embodiments, the clothes cleaning system may include an antimicrobial unit disposed in the storage container. The antimicrobial unit may be a variety of components and combinations of components. The antimicrobial unit may be configured to neutralize or limit the growth of microbes within the storage chamber. The antimicrobial unit may be configured to neutralize or limit the growth of microbes within the air and on items within the storage chamber.

In some embodiments, the antimicrobial unit includes a dehumidifier. The dehumidifier may be coupled to the storage container. The dehumidifier may have a dehumidifier inlet and a dehumidifier outlet. The dehumidifier may be configured to receive air from the storage chamber through the dehumidifier inlet, remove moisture from the air, and then return the air to the storage chamber through the dehumidifier outlet. In various embodiments the dehumidifier removes moisture from the air by directing the air over a refrigerated evaporator with a fan. In embodiments where there are multiple sections of the storage chamber, the dehumidifier may be configured to dehumidify air in multiple sections within the storage chamber. As such, the dehumidifier may have multiple dehumidifier inlets, one dehumidifier inlet in one section and another dehumidifier inlet in another section, for example. In some embodiments the dehumidifier may be configured to dehumidify a single section within a storage chamber. Advantageously, the dehumidifier may reduce moisture and humidity in the storage chamber to slow or cease microbial growth.

The water that is extracted from the air by the dehumidifier may be one or more of: (i) contained by the dehumidifier itself, (ii) transferred to a storage container within the storage chamber, (iii) transferred to a location outside of the storage container, or (iv) transferred to another component for use. When the water is transferred to a location outside of the storage container, the housing of the storage container may define a liquid drain through which the collected water may pass. When the water is transferred to another component for use, the component may be an air washer such as an air washer of the first filtration assembly or the second filtration assembly. In such examples, a liquid conduit may extend from the dehumidifier to the other component to fluidly couple the dehumidifier and the other component. For example, a liquid conduit may fluidly couple the dehumidifier and the first filtration assembly. In another example, a liquid conduit may fluidly couple the dehumidifier and the second filtration assembly. Such a configuration may advantageously conserve water. Furthermore, having a water source within the storage container may result in a less complex construction of a storage container compared to a configuration where the water source (such as a home plumbing system) is outside the storage container and must be routed into the storage container.

The antimicrobial unit may include an ultraviolet (UV) light source. The UV light source may be disposed in the storage container. The UV light source may be configured to expose the storage chamber to UV light. In some embodiments, the UV light source may be configured to expose one or more sections of the storage chamber to UV light. The UV light may advantageously disinfect items such as clothes in the storage chamber. The UV light may advantageously neutralize or limit the growth of microbes by destroying nucleic acids and disrupting the deoxyribonucleic acid (DNA) of the microorganisms. The UV light source may be coupled to the housing.

The UV light source may emit UV-C light. The UV light source may emit UV light at a wavelength from 240 to 290 nanometers, more preferably from 250 to 280 nanometers, and even more preferably from 260 to 275 nanometers.

The antimicrobial unit may include an electrostatic precipitator. The electrostatic precipitator may be disposed in the storage container, such as the electrostatic precipitator discussed above with reference to the second filtration assembly. However, the electrostatic precipitator may be separate from the second filtration assembly in some embodiments. In embodiments, an electrostatic precipitator may generate ozone through the course of use. Because it may be undesirable to introduce ozone into the outside of the storage container, the electrostatic precipitator may be positioned such that such ozone is generated inside the storage chamber. The ozone may advantageously neutralize or limit the growth of microbes in the storage chamber. The ozone may also advantageously eliminate odors within the storage chamber.

To avoid or reduce the introduction of ozone into the outside environment, in various embodiments the storage container is configured to allow ozone generated by the electrostatic precipitator to decompose before allowing release of the air inside the storage chamber to the outside environment. For example, in some embodiments the first filtration assembly is disabled until the level of ozone within the storage chamber is below a threshold. Embodiments consistent with such a configuration is discussed in more detail below.

The storage container may incorporate a variety of features to enable autonomous, remote operation, or both autonomous operation and remote operation. For example, the storage container may have a first air monitoring system in gaseous communication with the storage chamber. The first air monitoring system may have a number of different sensors and components to help monitor air quality inside the storage chamber. In some embodiments, the first air monitoring system is configured to identify gaseous constituents in the storage chamber.

In some embodiments, the first air monitoring system is in operative communication with the clothes cleaning system and is configured to execute cleaning functions of the clothes cleaning system in accordance with the gaseous constituents identified by the first air monitoring system. In this context the first air monitoring system may define patterns of identified pollutants to assign them to a specific cleaning action. In particular, the first air monitoring system may be configured to specify the clothes cleaning operations that the clothes cleaning system will undertake.

Components of the clothes cleaning system may be activated by the first air monitoring system to clean the clothes consistently with the identified gaseous constituents. For example, the conduit opening of the airflow conduit may be automatically opened upon identification by the first air monitoring system of particular gaseous constituents that may signal that ventilation is required or desirable. Identification of particular gaseous constituents may be the identification of a minimum level of one or more particular gaseous constituents in the air in the storage chamber.

The storage container may incorporate a first filtration assembly communication module coupled to the storage container. The first filtration assembly communication module may be in data communication with the first air monitoring system. The first filtration assembly communication module may have a wireless communication component configured to communicate with a user device such as a laptop or smartphone.

The first filtration assembly communication module may send data directly to a user device or to a database that may be accessed by a user through a network. The data may include the operational status of the first filtration assembly. The data may reflect the air quality status as measured by the first air monitoring system. The data may reflect the gaseous constituents identified in the storage chamber, such as ozone. The first filtration assembly communication module may inform a user of the status of the clothes cleaning system. For example, in some embodiments the first filtration assembly communication module sends data to a user device indicating that the items in the storage container, such as clothing, is considered to be clean.

The first filtration assembly communication module may be configured to receive operational instructions from the user device to selectively operate storage container components such as components of the clothes cleaning system and the first filtration assembly.

In some embodiments, the first air monitoring system may include an ozone sensor to detect the level of ozone within the storage chamber. In such embodiments, the ozone sensor may be disposed in the storage chamber. In embodiments, the ozone sensor may be positioned in a section of the storage chamber where the ozone is generated. In embodiments, the ozone sensor may be positioned adjacent to the first filtration assembly. In embodiments, the ozone sensor may be positioned upstream of the housing outlet. In embodiments, the ozone sensor may be positioned adjacent the secondary outlet of the airflow conduit.

In a number of embodiments, the ozone sensor is in operative communication with the first filtration assembly. The ozone sensor may be configured to disable operation of the first filtration assembly upon sensing a minimum level of ozone. The ozone sensor may be configured to enable operation of the first filtration assembly upon sensing a maximum level of ozone in the storage chamber. In particular, the ozone sensor may be in data communication with a controller that disables the first filtration assembly upon the ozone sensor sensing a minimum level of ozone. In some embodiments, the minimum level of ozone is 0.05 parts per million. In some embodiments, the minimum level of ozone is 0.10 parts per million. The controller may be configured to enable operation of the first filtration assembly upon the ozone sensor sensing a maximum level of ozone within the storage chamber.

The second filtration assembly of the storage container may incorporate a second air monitoring system configured to receive and monitor air from the second filtration inlet. The second air monitoring system may sense the air quality of the air entering the second filtration assembly from the outside environment and, based on the sensed quality, selectively operate components of the second filtration assembly using a controller. The sensed quality may be determined by identifying gaseous constituents, in some example. For example, if the second air monitoring system identifies gaseous constituents reflective of a minimum level of smoke or pollen in the air, the second air monitoring system may send a signal to a controller to operate an electrostatic precipitator to filter the incoming air.

The storage container may have a second filtration assembly communication module that is coupled to the storage container. The second filtration assembly communication module is configured to be in data communication with the second filtration assembly monitoring system. The second filtration assembly communication module may have a wireless communication component configured to communicate with a user device or a database accessible by a user device. The user device may be a laptop or smartphone, as examples. The second filtration assembly communication module may inform a user of air quality data received from the second air monitoring system. The second filtration assembly communication module may be configured to receive operational instructions from the user device to selectively operate storage container components such as components of the clothes cleaning system and the second filtration assembly.

Below, there is provided a non-exhaustive list of non-limiting examples. Any one or more of the features of these examples may be combined with any one or more features of another example, embodiment, or aspect described herein.
Example Ex1 A storage container comprising (i) a housing containing a storage chamber isolated from an outside environment, the housing defining a housing inlet and a housing outlet; and (ii) a first filtration assembly coupled to the housing about the housing outlet, wherein the first filtration assembly has a first filtration inlet configured to receive air from the storage chamber, a first filtration outlet configured to release air into the outside environment, and first filter media between the first filtration inlet and the first filtration outlet, wherein the storage container is configured to clean the air in the outside environment.
Example Ex2 The storage container of example Ex1, further comprising a second filtration assembly coupled to the housing about the housing inlet, wherein the second filtration assembly has a second filtration inlet configured to receive air from the outside environment, a second filtration outlet configured to release air into the storage chamber, and second filter media between the second filtration inlet and the second filtration outlet.
Example Ex3 The storage container of example Ex1 or Ex2, further comprising a clothes hanging structure configured to receive hanging clothing.
Example Ex4 The storage container of example Ex3, wherein the clothes hanging structure comprises a hanging rod extending across at least a portion of the storage chamber, wherein the hanging rod is configured to receive hangers of clothing.
Example Ex5 The storage container of any one of examples Ex1-Ex4, further comprising a clothes cleaning system in the storage chamber, the clothes cleaning system configured to execute cleaning functions within the storage chamber.
Example Ex6 The storage container of example Ex5, wherein the clothes cleaning system comprises a vertical air current generator configured to generate vertical, laminar airflow through the storage chamber.
Example Ex7 The storage container of example Ex5 or Ex6, wherein the clothes cleaning system comprises a clothes hanging structure configured to receive hanging clothing; and a vibration generator mechanically coupled to the clothes hanging structure.
Example Ex8 The storage container of example Ex7, wherein the clothes cleaning system further comprises a debris collector positioned below the clothes hanging structure.
Example Ex9 The storage container of any one of examples Ex5-Ex8, wherein the clothes cleaning system comprises a dehumidifier having a dehumidifier inlet configured to receive air from the storage chamber.
Example Ex10 The storage container of example Ex9, further comprising a liquid conduit fluidly coupling the dehumidifier and the first filtration assembly, and wherein the first filtration assembly comprises an air washer.
Example Ex11 The storage container of any one of examples Ex5-Ex10, wherein the clothes cleaning system comprises an antimicrobial unit disposed in the storage container.
Example Ex12 The storage container of example Ex11, wherein the antimicrobial unit comprises an ultraviolet (UV) light source disposed in the storage container.
Example Ex13 The storage container of example Ex11 or Ex12, wherein the antimicrobial unit comprises an electrostatic precipitator disposed in the storage container.
Example Ex14 The storage container of any one of examples Ex5-Ex13, wherein the clothes cleaning system comprises an airflow conduit extending through a length of the storage chamber, wherein the airflow conduit has a conduit inlet configured to receive air from the second filtration outlet and the airflow conduit has a plurality of openings that cumulatively define a conduit outlet along the length of the storage chamber.
Example Ex15 The storage container of any one of example Ex2-Ex14 comprising the second filtration assembly, wherein the second filtration assembly comprises an electrostatic precipitator.
Example Ex16 The storage container of any one of examples Ex2-Ex15 comprising the second filtration assembly, wherein the second filtration assembly comprises a second filtration assembly monitoring system configured to receive and monitor air from the second filtration inlet.
Example Ex17 The storage container of any one of examples Ex2-Ex16 comprising the second filtration assembly, further comprising a second filtration assembly communication module coupled to the storage container, the second filtration assembly communication module in data communication with the second filtration assembly monitoring system and the second filtration assembly communication module comprising a wireless communication component configured to communicate with a user device.
Example Ex18 The storage container of any one of examples Ex1 to Ex17, further comprising an ozone sensor disposed in the storage chamber.
Example Ex19 The storage container of example Ex18, wherein the ozone sensor is in operative communication with the first filtration assembly and is configured to disable operation of the first filtration assembly upon sensing a minimum level of ozone.
Example Ex20 The storage container of any one of examples Ex2-Ex19 comprising the second filtration assembly, wherein the second filtration assembly comprises a second air monitoring system.
Example Ex21 The storage container of any one of examples Ex1 to Ex20, further comprising a first air monitoring system in gaseous communication with the storage chamber.
Example Ex22 The storage container of example Ex21, wherein the first air monitoring system is configured to identify gaseous constituents in the storage chamber.
Example Ex23 The storage container of example Ex22, wherein the first air monitoring system is in operative communication with the clothes cleaning system and is configured to execute cleaning functions of the clothes cleaning system in accordance with the gaseous constituents identified by the first air monitoring system.
Example Ex24 The storage container of example Ex22, further comprising a first filtration assembly communication module coupled to the storage container, wherein the first filtration assembly communication module is in data communication with the first air monitoring system and the first filtration assembly communication module comprises a wireless communication component configured to communicate with a user device.
Example Ex25 The storage container of any one of examples Ex1 to Ex24, wherein the storage container is electrically connectable to a home utility control system for selective operation of the storage container through the home utility control system.
Example Ex26 A method of simultaneously cleaning air in an outside environment comprising: (i) passing chamber air from a storage chamber of a housing through a first filtration assembly and through a housing outlet of the housing; and (ii) passing outside air from outside the housing through a housing inlet of the housing and a second filtration assembly into the storage chamber of the housing.
Example Ex27 The method of example Ex26, further comprising a dehumidifier dehumidifying air inside of the storage chamber.
Example Ex28 The method of example Ex27, further comprising the dehumidifier collecting water from the dehumidifying.
Example Ex29 The method of example Ex28, further comprising transferring water from the dehumidifier to the first filtration assembly, wherein the first filtration assembly comprises an air washer and the first filtration assembly uses the transferred water to filter the chamber air.
Example Ex30 A method of filtering air comprising: (i) dehumidifying chamber air in a storage chamber of a housing and collecting water from the dehumidifying; (ii) transferring the water from the dehumidifier to an air washer coupled to a housing outlet of the housing; (iii) washing the chamber air in the storage chamber by the air washer using the transferred water; and (iv) expelling air from the storage chamber through the housing outlet.
Example Ex31 The method of example Ex30, further comprising passing the chamber air through a first filtration assembly comprising the air washer.
Example Ex32 The method of example Ex31, further comprising passing outside air from outside the housing through a housing inlet of the housing and a second filtration assembly into the storage chamber of the housing.
Example Ex33 The method of any one of the examples Ex26 to Ex32, further comprising vibrating a hanging rod disposed in the housing.
Example Ex34 The method of example Ex33, further comprising collecting debris in a debris collector positioned below the hanging rod.
Example Ex35 The method of any one of the examples Ex26 to Ex34, further comprising exposing the storage chamber to an antimicrobial unit disposed within the chamber.
Example Ex36 The method of example Ex35, wherein the antimicrobial unit comprises UV light from a UV light source.
Example Ex37 The method of any one of the examples Ex26 to Ex36, further comprising directing outside air from the second filtration assembly through an airflow conduit and out of a conduit outlet defined by a plurality of openings positioned along a length of the storage chamber.
Example Ex38 The method of any one of examples Ex26 to Ex37, further comprising generating ozone by the second filtration assembly.
Example Ex39 The method of example Ex38, further comprising detecting ozone by an ozone sensor disposed in the storage chamber and disabling operation of the first filtration assembly upon detection of a minimum level of ozone.
Example Ex40 The method of example Ex39, further comprising sending data sensed by the ozone sensor to a user device.
Example Ex41 The method of any one of examples Ex26 to Ex40, further comprising identifying gaseous constituents in the storage chamber by a first air monitoring system and execute cleaning functions in accordance with the constituents identified by the first air monitoring system.

Examples will now be further described with reference to the figures in which:

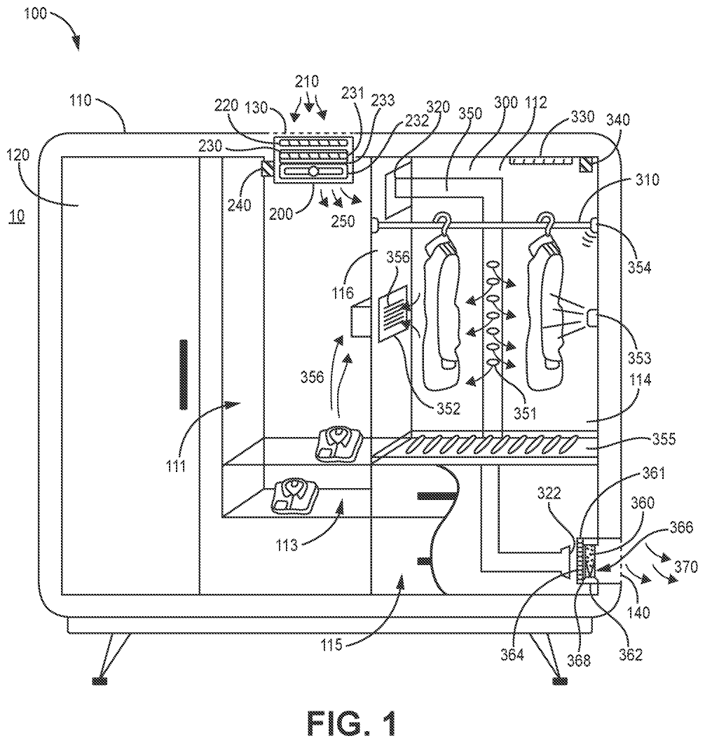

FIG. 1 shows is an example storage container consistent with the invention.

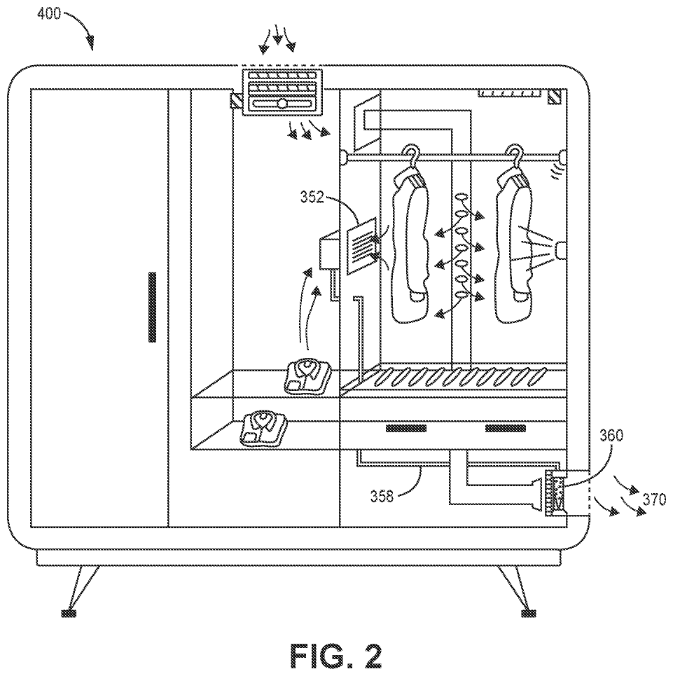

FIG. 2 shows is another example storage container consistent with the invention.

FIG. 1 depicts one example storage container 100 consistent with the present application that is configured to filter the air in the outside environment 10, such as a room, within which the storage container 100 is situated. The storage container 100 has a housing 110 and a storage chamber 112 isolated from an outside environment 10. The storage container 100 has a housing inlet 130 and a housing outlet 140. Air from the outside environment 10 may pass into the storage chamber 112 through the housing inlet 130. Air from the storage chamber 112 may pass into the outside environment 10 through the housing outlet 140. The storage container is configured to clean the air in the outside environment and clean items stored in the storage chamber 112. The storage container has a first filtration assembly 360 coupled to the housing 110 about the housing outlet 140.

The first filtration assembly 360 is configured to filter air from the storage chamber 112 and release the filtered air into the outside environment 10. In particular, the first filtration assembly 360 has a first filtration inlet 364 that is configured to receive air from the storage chamber 112. The first filtration assembly 360 has a first filtration outlet 366 that is configured to release air into the outside environment 10. The first filtration assembly 360 has first filter media 368 between the first filtration inlet 364 and the first filtration outlet 366. As such, air from the storage chamber 112 passes through the first filter media 368 before passing through the first filtration outlet 366. The first filtration assembly 360 of the storage container 100 is configured to purify the air in the storage container 100 and then release that air to the outside environment 10.

The first filtration assembly 360 may have a variety of different configurations and components as has been discussed in detail above. The first filter media 368 may be a liquid such as water or a water solution. In some embodiments, the first filtration assembly 360 has first filter media 368 that is a textile. The textile can be, for example, a woven cotton fabric. The first filtration assembly 360 may also incorporate components that are configured to purify the air such as, for example, a UV light source 362.

The storage container 100 has a second filtration assembly 230 coupled to the housing 110 about the housing inlet 130. The second filtration assembly 230 has a second filtration inlet 210, a second filtration outlet 250, and second filter media 231 between the second filtration inlet 210 and the second filtration outlet 250. The second filtration inlet 210 is configured to receive air from the outside environment 10. The second filtration outlet 250 is configured to release air into the storage chamber 112. A fan 232 transmits the air from the second filtration inlet 210 to the second filtration outlet 250. As such, air from the outside environment 10 passes through the second filter media 231 before passing through the second filtration outlet 250.

The second filtration assembly 230 may have a variety of different configurations and components, as has been discussed above. Here, the second filtration assembly 230 incorporates second filter media 231. The second filter media 231 may include a carbon filter media or HEPA (high efficiency particle air) filter media, in some examples. The second filtration assembly 230 may also incorporate other components that are configured to purify the air such as, for example, a UV light source or an electrostatic precipitator 233. The air purification of the second filtration assembly 230 advantageously exposes the storage chamber 112 and the items within the storage chamber 112 to relatively clean air compared to the air from the outside environment 10.

The storage container 100 may have a variety of different configurations. The storage container 100 has a storage chamber 112 and in examples such as FIG. 1, the storage chamber 112 has multiple storage sections 111, 113, 114, 115. In some embodiments, one or more storage sections, such as a first storage section 111 and second storage section 113, may define shelving to receive items. One or more storage sections, such as a third storage section 114, may be used for storing hanging clothes. In some embodiments, a fourth storage section 115 may include one or more drawers configured to contain stored items. Other combinations of storage sections are certainly contemplated.

The storage container 100 has a clothes hanging structure 310 to receive hanging clothing. In examples, the clothes hanging structure 310 is a hanging rod extending across at least a portion of the storage chamber 112. Particular to this example, the hanging rod 310 extends across the third storage section 114 from the housing 110 to a divider wall 116. The divider wall 116 separates a first storage section 111 from the third storage section 114. The hanging rod 310 is configured to receive hangers of clothing. In some other embodiments the clothes hanging structure may be one or more hooks configured to receive handing clothing. The hooks may, for example, be coupled to the storage container 100 within the storage chamber 112. The hooks may be coupled to the housing 110.

The storage container 100 has a user access point from which to access the contents of the storage chamber 112. In various embodiments the user access point is a container door 120 coupled to the storage container 100. The container door 120 may be slidable, pivotable, or extendable across the storage chamber 112 to selectively isolate the storage chamber 112 from the outside environment 10. When in a closed position, the container door 120 generally restricts air circulation between the storage chamber 112 and the outside environment 10.

The storage container 100 may generally be configured to clean items stored in the storage chamber 112. This may be accomplished through a variety of approaches. The storage container 100 may have a clothes cleaning system within the storage chamber 112 that is configured to execute cleaning functions in the storage chamber 112. The clothes cleaning system may have a variety of different components and combinations of components. Example components of clothes cleaning systems will now be described.

In some embodiments consistent with the current example, the clothes cleaning system may include a vibration generator 354. The vibration generator 354 may be mechanically coupled to the clothes hanging structure 310. In some embodiments the vibration generator 354 is configured to generate vibrations and transmit the vibrations to the clothes hanging structure 310. In some embodiments the vibration generator 354 is configured to generate ultrasonic vibrations. The vibrations are transmitted to the clothes hanging structure 310, which results in vibration of the clothes hanging on the clothes hanging structure 310. The vibration of the clothes may result in advantageously shaking debris from the clothing. The storage container 100 has a debris collector 355 is positioned to receive shaken debris from the clothing on the clothes hanging structure 310. In particular, the debris collector 355 is positioned vertically below the clothes hanging structure 310 such that gravity transports the shaken debris from the clothing to the debris collector 355.

In some embodiments, the clothes cleaning system has an airflow conduit 350 that is configured to transmit purified air to a section of the storage chamber 112. The airflow conduit 350 extends through a length of the storage chamber 112. The airflow conduit 350 has a conduit inlet 320 configured to receive air from the second filtration outlet 250. The airflow conduit 350 has a conduit outlet 351 that is cumulatively defined by a plurality of openings along the length of the storage chamber 112. In the current example, the plurality of openings defining the conduit outlet 351 extend along a substantial portion of the length of the third storage section 114, where the length is substantially perpendicular to the hanging rod 310. The hanging rod 310 extends across the width of the third storage section 114.

In various embodiments, the conduit outlet 351 is selectively openable. The conduit outlet may advantageously allow for selective ventilation of the relevant section 114 of the storage chamber 112. In some embodiments the conduit outlet 351 may be operated by a user, such as with a smartphone, and in some embodiments the conduit outlet 351 may be selectively opened and closed autonomously by the system, which will be described below. In some embodiments pulsed air may be released through the plurality of openings defining the conduit outlet 351, which may advantageously shake debris from items adjacent the conduit outlet 351.

In examples, the airflow conduit 350 extends between the second filtration assembly 230 and the housing outlet 140. In particular, the airflow conduit 350 extends from the dividing wall 116 adjacent to the housing outlet 140 or, more particularly, adjacent to the first filtration assembly 360. The airflow conduit 350 defines a secondary outlet 322 adjacent to the first filtration assembly 360. As such, when the conduit outlet 351 is closed, air enters the airflow conduit 350 through the conduit inlet 320 and exits the airflow conduit 350 through the secondary outlet 322, at which point the air exiting the airflow conduit 350 may extend through the first filtration assembly 360 to outside environment 10 of the storage container 100. In some embodiments the secondary outlet 322 may be omitted.

In various embodiments, the clothes cleaning system includes an antimicrobial unit disposed in the storage container 100. The antimicrobial unit may be a variety of components and combinations of components. The antimicrobial unit is generally configured to neutralize or limit the growth of microbes within the storage chamber.

In some embodiments, the antimicrobial unit includes a dehumidifier 352. The dehumidifier 352 may be coupled to the storage container 100. The dehumidifier 352 has a dehumidifier inlet 356 that is configured to receive air from the storage chamber 112, remove moisture from the air, and then return the air to the storage chamber 112. In various embodiments the dehumidifier 352 removes moisture from the air by directing the air over a refrigerated evaporator with a fan. In this example, the dehumidifier 352 is configured to dehumidify air in multiple sections within the storage chamber 112 including the first storage section 111 and the third storage section 114. As such, in the current example, the dehumidifier 352 has multiple dehumidifier inlets 356, one in the first storage section 111 and another in the third storage section 114. In some embodiments the dehumidifier may dehumidify a single section within a storage chamber 112. Advantageously, the dehumidifier 352 may reduce moisture and humidity in the storage chamber 112 to slow or cease microbial growth.

The water that is collected from the air by the dehumidifier may be contained by the dehumidifier itself, drained to a container within the storage chamber, or drained to a location outside of the storage container 100. In the latter example, the storage container would have a drain through which the collected water may be drained. An alternate example will also be described below with reference to FIG. 2, where the water collected by the dehumidifier is recycled by the storage container for filtration operations.

In various embodiments, the antimicrobial unit may include an ultraviolet (UV) light source 353 disposed in the storage container 100. The UV light source 353 is configured to expose the storage chamber 112 to UV light. The UV light may advantageously disinfect items such as clothes in the storage chamber 112. The UV light may advantageously neutralize or limit the growth of microbes by destroying nucleic acids and disrupting the deoxyribonucleic acid (DNA) of the microorganisms.

In embodiments, the antimicrobial unit may include an electrostatic precipitator disposed in the storage container 100, such as the electrostatic precipitator 233 discussed above with reference to the second filtration assembly 230. In some embodiments the electrostatic precipitator 233 may be separate from the second filtration assembly 230. In a variety of embodiments, an electrostatic precipitator 233 generates ozone through the course of use, and such ozone may be used by the storage container to neutralize or limit the growth of microbes. The ozone may also further clean items stored in the storage container 100 such as by eliminating odors. In various embodiments the system is configured to allow the ozone to decompose before releasing the air inside the storage chamber 112 to the outside environment 10 via the first filtration assembly 360.

The storage container 100 may incorporate a variety of features to enable autonomous operation, remote operation, or both autonomous operation and remote operation. For example, the storage container 100 may have a first air monitoring system 330 in gaseous communication with the storage chamber 112. The first air monitoring system 330 may have a number of different sensors and components to help monitor air quality inside the storage chamber 112. In some embodiments, the first air monitoring system 330 is configured to identify gaseous constituents in the storage chamber 112.

In some embodiments, the first air monitoring system 330 is in operative communication with the clothes cleaning system and is configured to execute cleaning functions of the clothes cleaning system in accordance with the gaseous constituents identified by the first air monitoring system 330. In this context the first air monitoring system 330 may define patterns of identified pollutants to assign them to a specific cleaning action. In particular, the first air monitoring system may be configured to specify the clothes cleaning operations that the clothes cleaning system will undertake. Components of the clothes cleaning system may then be activated by the first air monitoring system 330 to clean the clothes adequately. For example, the conduit outlet 351 of the airflow conduit 350 may be opened upon identification by the first air monitoring system that ventilation is required.

The storage container 100 may incorporate a first filtration assembly communication module 340 coupled to the storage container 100. The first filtration assembly communication module 340 is in data communication with the first air monitoring system 330. The first filtration assembly communication module 340 has a wireless communication component configured to communicate with a user device such as a laptop or smartphone. The first filtration assembly communication module 340 may send data such as the operational status of the first filtration assembly or the air quality status as measured by the first air monitoring system 330.

The first air monitoring system has an ozone sensor 361 to detect the level of ozone within the storage chamber 112. The ozone sensor 361 is generally disposed in the storage chamber 112. In a number of embodiments, the ozone sensor 361 is in operative communication with the first filtration assembly 360 and is configured to disable operation of the first filtration assembly 360 upon sensing a minimum level of ozone. The ozone sensor 361 may be configured to enable operation of the first filtration assembly 360 upon sensing a maximum level of ozone in the storage chamber 112. In particular, the ozone sensor 361 may be in data communication with a controller that disables the first filtration assembly 360 upon the ozone sensor sensing a minimum level of ozone. The controller may be configured to enable operation of the first filtration assembly 360 upon the ozone sensor sensing a maximum level of ozone within the storage chamber 112.

The second filtration assembly 230 of the storage container 100 may incorporate a second air monitoring system 220 configured to receive and monitor air from the second filtration inlet 210. The second air monitoring system 220 may sense the air quality of the air entering the second filtration assembly 230 from the outside environment 10 and, based on the sensed quality, selectively operate components of the second filtration assembly 230.

The storage container 100 has a second filtration assembly communication module 240 that is coupled to the storage container 100. The second filtration assembly communication module 240 is configured to be in data communication with the second air monitoring system 220. The second filtration assembly communication module 240 has a wireless communication component configured to communicate with a user device. The second filtration assembly communication module 240 may inform a user of air quality data received from the second air monitoring system 220. The second filtration assembly communication module 240 may inform a user of the status of the clothes cleaning system. For example, in some embodiments the second filtration assembly communication module 240 sends data to a user device indicating that the items in the storage container, such as clothing, is considered to be clean.

FIG. 2 depicts another example storage container 400 where the water extracted from the storage container air by the dehumidifier 352 is used by the storage container 400 for filtration operations. Such a configuration may advantageously conserve water. Furthermore, having a water source within the storage container 400 may result in a less complex construction of a storage container compared to a configuration where the water source (such as a home plumbing system) is outside the storage container and must be routed into the storage container.

As discussed above, in such an embodiment the filtration operations may be associated with an air washer. In the current example, the first filtration assembly 360 may have an air washer and a liquid conduit 358 may fluidly couple the dehumidifier 352 to the air washer. The storage container 400 is similar to the storage container 100 discussed above with reference to FIG. 1.

For the purpose of the present description and of the appended claims, except where otherwise indicated, all numbers expressing amounts, quantities, percentages, and so forth, are to be understood as being modified by the term “precisely” or “about”. In the context of “about”, a number A is generally understood as A±5 percent or less of A. For example, a number A may be A±3 percent or less of A, such as A±2 percent or less of A. Within this context, a number A may be considered to include numerical values that are within general standard error for the measurement of the property that the number A modifies. The number A, in some instances as used in the appended claims, may deviate by the percentages enumerated above provided that the amount by which A deviates does not materially affect the basic and novel characteristic(s) of the claimed invention. Also, all ranges include the maximum and minimum points disclosed and include any intermediate ranges therein, which may or may not be specifically enumerated herein.

1-15. (canceled)

16. A storage container comprising:
a housing containing a storage chamber isolated from an outside environment, the housing defining a housing inlet and a housing outlet;
a first filtration assembly coupled to the housing about the housing outlet, wherein the first filtration assembly has a first filtration inlet configured to receive air from the storage chamber, a first filtration outlet configured to release air into the outside environment, and first filter media between the first filtration inlet and the first filtration outlet, wherein the storage container is configured to clean the air in the outside environment;
a second filtration assembly coupled to the housing about the housing inlet, wherein the second filtration assembly has a second filtration inlet configured to receive air from the outside environment, a second filtration outlet configured to release air into the storage chamber, and second filter media between the second filtration inlet and the second filtration outlet;
a first air monitoring system in gaseous communication with the storage chamber; and
a second air monitoring system configured to receive and monitor air from the second filtration inlet.

17. The storage container of claim 16, further comprising a clothes cleaning system in the storage chamber, the clothes cleaning system configured to execute cleaning functions within the storage chamber.

18. The storage container of claim 17, wherein the clothes cleaning system comprises a clothes hanging structure configured to receive hanging clothing; and
a vibration generator mechanically coupled to the clothes hanging structure.

19. The storage container of claim 17, wherein the clothes cleaning system comprises a dehumidifier having a dehumidifier inlet configured to receive air from the storage chamber.

20. The storage container of claim 19, further comprising a liquid conduit fluidly coupling the dehumidifier and the first filtration assembly, and wherein the first filtration assembly comprises an air washer.

21. The storage container of claim 17, wherein the clothes cleaning system comprises an antimicrobial unit disposed in the storage container.

22. The storage container of claim 21, wherein the antimicrobial unit comprises an electrostatic precipitator disposed in the storage container.

23. The storage container of claim 17, wherein the clothes cleaning system comprises an airflow conduit extending through a length of the storage chamber, wherein the airflow conduit has a conduit inlet configured to receive air from the second filtration outlet and the airflow conduit has a plurality of openings that cumulatively define a conduit outlet along the length of the storage chamber.

24. The storage container of claim 16, wherein the second filtration assembly comprises an electrostatic precipitator.

25. The storage container of claim 16, further comprising an ozone sensor disposed in the storage chamber.

26. The storage container of claim 25, wherein the ozone sensor is in operative communication with the first filtration assembly and is configured to disable operation of the first filtration assembly upon sensing a minimum level of ozone.

27. The storage container of claim 16, wherein the first air monitoring system is configured to identify gaseous constituents in the storage chamber.

28. The storage container of claim 16, further comprising a first filtration assembly communication module coupled to the storage container, wherein the first filtration assembly communication module is in data communication with the first air monitoring system and the first filtration assembly communication module comprises a wireless communication component configured to communicate with a user device.
